(19) United States
(12) Patent Application Publication
(10) Pub. No.: US 2026/0151212 A1
(43) Pub. Date: Jun. 4, 2026

(54) METHOD FOR PRODUCING A DENTAL PROSTHESIS

(71) Applicant: SNAP-CONE-PROSTHETICS UG (HAFTUNGSBESCHRÄNKT), Seeheim-Jugenheim (DE)

(72) Inventor: Holger ZIPPRICH, Seeheim-Jugenheim (DE)

(21) Appl. No.: 19/122,763

(22) PCT Filed: Oct. 12, 2023
(86) PCT No.: PCT/EP2023/078340

§ 371 Date: Apr. 21, 2025

(30) Foreign Application Priority Data
Oct. 21, 2022 (DE) 10 2022 127 938.3

(51) Int. Cl. A61C 5/77; A61K 6/802; A61K 6/844

(52) U.S. Cl. CPC A61C 5/77; A61K 6/802; A61K 6/844

(57) ABSTRACT

A method for manufacturing a dental prosthesis (2), which is provided with a number of matrix elements (18) corresponding to one of the matrix elements (14) in each case for removable fixation on a number of teeth (6) crowned with a respective male element (14) in the mouth of a patient, should be designed for a particularly high accuracy of fit of the dental prosthesis (2) using methods which are kept as simple as possible. According to the invention, multi-part matrix elements (18) are used for this purpose, each comprising an inner cap (32) which can be slipped onto the associated male element (14) and an outer cap (34) which is attached to the dental prosthesis (2), wherein in the space between the inner and outer caps (32, 34), an intermediate body (36) made of thermoplastic material connecting them to one another being arranged in the space between the inner and outer caps (32, 34), the intermediate body (36) being heated to a temperature above its softening temperature and thus being made deformable in order to align the inner cap (32) precisely relative to the outer cap (34) of the respective matrix element (18), and then, using the deformability of the intermediate body (36), the inner cap (32) is aligned relative to the outer cap (34) with regard to an optimized accuracy of fit, taking into account intraoral data reflecting the actual dentition situation in the oral cavity of the patient, before the relative position of the inner cap (32) set in this way relative to the outer cap (34) is preserved as a result of the solidification of the intermediate body (36) occurring during the subsequent cooling of the intermediate body (36).

The invention relates to a method for manufacturing a dental prosthesis which is provided with a number of matrix elements corresponding to one of the matrix elements for removable fixation on a number of teeth crowned with a respective male element in the mouth of a patient.

If teeth are lost, the aim is usually to replace them or at least close the gaps left by the loss. This can be done using so-called bridges, for example. In this case, the teeth adjacent to the gap are ground down and a crown is made to replace the lost teeth with corresponding bridge units. The bridges are mechanically anchored to the ground teeth. A pendant can also be attached to a bridge to treat a free-end situation. Such a situation can also be treated with removable clasp dentures, which are usually supported by natural teeth.

In addition to bridgework, lost teeth can also be replaced using endosseous implants. In this case, threaded posts are usually used as artificial tooth roots onto which the prosthetics can be anchored. This anchorage can also be fixed (screwed, cemented or bonded) or removable. In the anchoring of removable solutions, the adhesive forces that hold the prosthesis in position are usually based on a negative pressure effect, friction and/or retention.

As soon as too few teeth remain in the patient's mouth, other solutions are usually used. One option is to remove the entire remaining dentition and use mucosa-supported prostheses. These are inexpensive, but are not very comfortable for the patient to chew and are usually accompanied by bone loss in the upper and lower jaw.

The residual dentition and/or endosseous implants are often used as abutments for removable restorations. There are also a large number of possible solutions. Clasp prostheses, electroforming techniques on implants or implant bars, conical clasp systems, ball head anchors, silicone-metal matrix-patrix systems and other systems can be used.

In particular, so-called telescopic systems can be used, in which a dental prosthesis is removably attached to so-called telescopic crowns. These telescopic crowns are double crowns in which a “lower” or base crown, also known as a “primary crown”, in particular a patrix element, is firmly attached to a suitably prepared, for example ground, tooth in the manner of a conventional crowning. On its upper side, this patrix element forms a contact pin to which an “upper” crown segment or matrix element, also known as a “secondary crown”, can be detachably or removably attached, for example clicked on or clipped on. The crown segments thus form a type of matrix/patrix system. The upper crown segment, which forms the matrix, then serves as a support for the prosthetics, whereby the removable connection of the crown segments to each other makes the prosthesis removable as a whole. Parallel or conical telescopes are commonly used, which can be cast, milled or designed as a gold galvano prosthesis.

In the manufacture of all these systems, the patient's oral situation is usually recorded in a first step, for example by taking an impression with suitable impression material. The oral situation is then usually transferred to a physical, e.g. plaster, or virtual model. Using this model, the dental technician can then fabricate the prosthetics to suit the situation and requirements so that they can later be fitted as accurately as possible in the patient's mouth. The accuracy of fit is a very important factor here, as a high degree of accuracy is a prerequisite for a tight fit of the prosthesis in the patient's mouth, for example. Furthermore, a high accuracy of fit naturally also requires a high level of wearing comfort for the patient, so that they can feel as comfortable as possible with the prosthesis. The more accurately the prosthesis is made, the tighter the fit in the patient's mouth can be, which in turn leads to a very high level of wearing and chewing comfort. In contrast, the more flexible the removable fixation is, the more spongy the fit in the patient's mouth. Although this simplifies the fabrication of the prosthesis and reduces the manufacturing costs, a particularly high accuracy of fit is usually aimed for when weighing up these aspects.

One problem that has emerged in this context is that the described transfer of the patient situation to a physical or virtual model can contribute significantly to undesirable fitting inaccuracies.

In contrast, in order to provide a very accurately fitting matrix-patrix system, the matrices anchored in the prosthesis can be cemented or glued in the patient's mouth to avoid inaccuracies of fit. However, this involves increased effort and correspondingly higher costs.

In addition, all these systems are subject to wear due to repeated removal and insertion in the patient's mouth, which also varies depending on the system. This should be kept to a minimum when designing new concepts.

Another task of removable restorations is that the adhesive forces should be reproducible and give the patient the feeling that they have fixed teeth, although the dentures should not get stuck when they are removed. This means that the bond strength should not be too high and should not or hardly be dependent on the previously exerted chewing force.

At present, matrix/patrix systems based on a gold electroformed matrix on a ceramic matrix are the most popular and best variant in terms of wearing comfort and balance in the areas of adhesion or adhesive force and wear in telescope systems of the type described. They achieve the highest accuracy of fit within the matrix/patrix system and therefore also have an optimum suction effect or negative pressure effect in terms of adhesive force and very low wear due to the high-precision fit. As the system is usually conical in the area of the patrix-matrix connection and rests occlusally, there is no conical self-locking in the system, which also prevents tilting during removal. The only disadvantage of these systems is the very complex manufacture and the very high price.

The reason for the high cost is as follows. The dentist takes an impression of the patient's situation. The dental technician creates a patient model. The patrices are acquired or purchased on natural teeth, in particular using the CAD-Cam method (or previously cast). The matrices are then fabricated or purchased partially prefabricated on implants. A metal framework is then fabricated. The ensemble is then sent to the dentist. The dentist fixes (screws, glues or cements) the patrix elements onto the teeth. He then cements or bonds the matrix elements into the framework. There is a risk of the matrix-patrix system sticking in the patient's mouth, which can lead to time-consuming removal. The ensemble is then sent back to the dental technician so that he can create the finished prosthesis on the framework. Only then is the prosthesis finished and can be finally fitted in the patient's mouth. The advantage, however, is that the inaccuracies of fit of the usually 4 matrix/patrix systems can be minimized as far as possible by transferring the model from the patient.

Attempts are currently being made to replace the gold Galvano matrix with a plastic matrix, usually milled or 3D-printed. The plastics PTFE, PEEK, PPS and plastics with similar properties are usually used.

German patent application No. 10 2022 127 865.4, filed with the same priority date and entitled “Telescopic crown”, describes a telescopic crown of the above-mentioned type which is significantly improved compared to the prior art, with which the disadvantages mentioned can be avoided and which allows the provision of a dental prosthesis with a particularly high accuracy of fit at a comparatively low cost. In this improved telescopic crown, the matrix element is made in several parts and comprises an inner cap that can be attached to the male element and an outer cap that can be attached to the dental prosthesis, with an intermediate body made of thermoplastic material connecting them being arranged in the space between the inner and outer caps.

This is based on the idea that a high accuracy of fit of the prosthetics in the patient's oral cavity can be achieved by only roughly adapting the components used to form the prosthetics to the patient's oral situation during the preparation phase, whereby the final adaptation and fine-tuning should only take place during insertion into the patient's oral cavity and in response to any restoring forces and the like that arise. To make this possible, one of the components, i.e. the matrix or the patrix, should be designed in such a way that final positioning and alignment only takes place during insertion into the oral cavity. For this purpose, the male part or preferably the female part should be designed in several parts so that a first part-in this case the outer cap of the female part—can already be firmly attached to the denture during preparation. The alignment of the inner cap relative to the outer cap and thus the final positioning should then take place during insertion in response to the forces occurring in the oral cavity. For this purpose, the inner cap of the female part can be connected to its outer cap via an intermediate body which, according to the concept of the invention, increasingly solidifies during or immediately after insertion, starting from an initially deformable state. The solidification should take place during insertion so that influences of the oral environment can also be absorbed and the components can align themselves automatically in an optimized manner in response to the oral situation. Subsequently, i.e. after solidification, the optimized alignment of the components should be suitably fixed.

For example, the inner cap could be connected to the outer cap via a cement or adhesive joint, which, with sufficient volume for the cement or adhesive, offers sufficient leeway for positioning optimization and alignment of the components as long as the cement or adhesive is in the solidification phase. Alternatively, the intermediate body can be made of thermoplastic material. During incorporation, the intermediate body can be suitably heated beforehand so that a certain softening and thus deformability occurs. The insertion can then be carried out in this state so that the inner and outer copings align with each other appropriately according to the fine details of the oral situation. On cooling, the intermediate body then solidifies again while retaining this geometry, so that the desired optimization in positioning is suitably fixed.

Said telescope system particularly and preferably has one or more of the following aspects:
This is a matrix-patrix system
The patrix is formed by a cap fixed on a natural tooth.
The female part is formed by a cap which is placed over the male part.
The die has a multi-part design and comprises an outer and an inner cap, between which a deformable, preferably thermoplastic intermediate body is arranged
For the patrix designed as a cap on a natural tooth, the prepared tooth determines the cross-section and the functional height. The preparation angle is advantageously 1° to 6°, particularly preferably 2° to 4°
The patrix as a cap on a natural tooth is preferably made of titanium or titanium alloy, zirconium or zirconium alloy, tantalum or tantalum alloy, a non-precious metal or a ceramic with a base of zirconium dioxide (ZrO2) and or aluminum oxide (Al₂O₃) and or a silicate ceramic or an alloy of the metals or a mixture of the ceramics
Adhesion/the adhesive force between male and female parts is based on a conical design (angle bisector=2°-10°; 2°-8°; 4°-6°) via suction effect
Adhesion/adhesive force between male part and female part is based on a conical design (2°-10°; 2°-8°; 4°-6°) via suction and additional retention by means of at least one circular undercut
The undercut is cut into the cone of the superstructure part as a circumferential bead and preferably as a circumferential groove
The undercut is positioned in the conical area. Preferably, it is positioned in the apical half, the apical third or the apical quarter of the conical portion.
The adhesion/adhesive force can, but does not have to be additionally supported by a conical self-locking device.

The male part and/or in particular the female part of the telescopic system improved in this way is thus designed as a multi-part system which, viewed in longitudinal section, can be understood as a multi-layer system, preferably a double-or triple-layer system. In an embodiment designed as a double-layer system or two-part system, this preferably consists of an outer cap made of a metallic base material or preferably of a plastic and particularly preferably of a biocompatible high-performance plastic, and the thermoplastic intermediate body facing the patrix. Preferably, both plastics are autoclavable, i.e. temperature-resistant up to at least 135° C. Preferably, the plastic facing the patrix is designed with regard to its choice of material for
low water absorption
High mechanical strength
High wear resistance

The plastic facing away from the male part and forming the outer cap could be softer than the thermoplastic facing the male part.

In one embodiment as a three-layer system or three-component system, the matrix element is designed in accordance with one aspect of the invention:
Like the double-layer system and additionally an inner cap facing the male part
the inner cap facing the prosthesis can be easily cemented/bonded to it- and is mechanically stronger than the other two resins
Good mechanical bonding with the plastic facing the male part.
Preferably made of titanium, zirconium, tantalum, an alloy of at least one of the metals.

The inner cap of the die element should be capable of being suitably tilted or displaced relative to the outer cap during final alignment, i.e. during curing of the thermoplastic intermediate body. Advantageously and according to an aspect of the invention, the inner cap is made of a plastic, preferably a high performance plastic. According to an aspect of the invention, this plastic should have its softening temperature significantly above the softening temperature of the thermoplastic intermediate body, preferably 20, 25, or even 50% higher. Furthermore, the plastic forming the inner cap should have a comparatively high abrasion resistance and mechanical strength, so that repeated removal and reinsertion of the telescopic crown is possible even without significant wear of these components. The inner cap is preferably made of PEEK or a comparable plastic.

The thermoplastic intermediate body provided according to this improvement can also be designed in the form of a layer, i.e. as a thermoplastic layer.

The application of such a thermoplastic layer can compensate for the inaccuracy of fit between the patient situation and the model situation at the dental technician.

The present invention is now based on the task of providing an improved method for manufacturing a dental prosthesis, with which a dental prosthesis can be manufactured with particularly high accuracy of fit in a simplified manner.

According to the invention, this task is solved by a method for manufacturing a dental prosthesis which is provided with a number of matrix elements corresponding to one of the matrix elements for removable fixation on a number of teeth crowned with a respective male element in the mouth of a patient, wherein the or each matrix element is each made in several parts and comprises an inner cap which can be slipped onto the associated male element and an outer cap which is attached to the dental prosthesis, and wherein an intermediate body made of thermoplastic material is arranged in the space between the inner cap and the outer cap and connects them to one another, in which the intermediate body is heated to a temperature above its softening temperature and thus made deformable in order to align the inner cap precisely relative to the outer cap of the respective matrix element, and then, using the deformability of the intermediate body, the inner cap is aligned relative to the outer cap with regard to an optimized accuracy of fit, taking into account intraoral data reflecting the actual dentition situation in the patient's oral cavity, before the relative position of the inner cap relative to the outer cap set in this way is conserved as a result of the solidification of the intermediate body occurring during the subsequent cooling of the intermediate body.

Alternatively, the said problem is solved according to one aspect of the invention with a method for producing a dental prosthesis which is provided with a number of matrix elements corresponding to one of the matrix elements in each case for removable fixation on a number of teeth crowned with a respective patrix element in the mouth of a patient, in particular using multi-part matrix elements which each comprise an inner cap which can be plugged onto the associated patrix element and an outer cap which is attached to the dental prosthesis, wherein an intermediate body of thermoplastic material connecting the inner and outer caps is arranged in the space between the inner and outer caps, wherein, on the basis of intraoral data reflecting the actual dentition situation in the patient's oral cavity, those matrix elements are selected from a number of basic matrix element types stored in a component library which permit reliable fixation of the prosthesis with the least possible removal of tooth structure, and wherein the required grinding of the teeth is determined on the basis of this selection and made available to the practitioner as instructions for action.

The invention is based on the consideration that a further improved and simplified manufacturing process should specifically utilize the possibilities and degrees of freedom provided by the aforementioned improved design of the telescopic crowns. In particular, the fact that the multi-part design of the matrix elements means that the fine alignment and adjustment of the components relative to each other is only planned at a relatively late stage anyway, i.e. when they are inserted into the patient's mouth, can be utilized. Until then, it is possible to work with relatively roughly prefabricated components. Among other things, this also allows the use of standard components, which can be easily provided in large quantities and are only aligned and positioned finely and in detail in the final work step, using the degrees of freedom made possible by the thermoplastic intermediate piece. Such standard components can thus be stored as basic types in a library and made available for selection.

According to aspects of the invention, the manufacturing process of the patrices on natural teeth including software and library is thus designed according to the following aspects and criteria:

The restoration of natural teeth intended as supporting abutments for prosthetics with corresponding patrices or prefabricated patrix caps represents a major challenge. The dentist has to prepare the tooth stumps in order to be able to use them as supporting abutments. It would first be necessary to prepare the teeth in such a way that a prefabricated patrix cap can be accurately fixed. The height, the cone angle and the cross-section of the ground tooth, i.e. the tooth as a whole, would have to be adapted as precisely as possible to the inner lumen of the patrix cap. In addition, the intended insertion angle of the prosthesis should also be taken into account when attaching the prosthesis to the supporting abutments. This is particularly important, as three to eight (preferably four to six) abutments would have to be prepared in such a way that the prosthesis can be inserted without tension after the patrix caps have been fixed.

The task given by this is solved in accordance with an aspect of the invention which is regarded as independently inventive, in that a virtual library of patrix designs is provided in the manner of basic types, which comprises ground support abutments in the corresponding regions (anterior tooth, canine tooth, posterior tooth, upper jaw and lower jaw in each case). It is advantageous if there are preferably a number of dimensions of the patrix dimensions per region. The dentist now only has to prepare the tooth stumps in such a way that one of the virtually provided library patrix caps with a sufficient material thickness would fit over this ground tooth stump. In a CAD-CAM process, it is then possible to position the patrix caps of all abutments on the ground tooth stumps in optimum alignment with each other and to manufacture them accordingly.

In order to make this possible, according to one aspect of the invention it is envisaged that the practitioner scans the teeth intended as supporting abutments in advance, if possible with the aid of an intraoral scanner (alternatively, a situation model could also be created using a conventional impression, which is then digitized with the aid of a scanner). Preferably, the entire jaw is scanned. Software then calculates for which patrix caps (for which prefabricated matrices should then be available) the practitioner must remove the least amount of tooth structure, i.e. grind the tooth or stump the least. If a complete intraoral scan is available, the software can also take into account the optimized insertion direction as described above. The dentist is then shown on a screen in the program on which tooth or stump he needs to remove, grind or grind away how much. The dentist can now start grinding. The dentist can carry out a new intraoral scan after completing the first grinding process as a check. This time, it is only necessary to record the ground tooth and the immediate surroundings in order to be able to make a statement about the shape achieved. If this is not sufficient, the software will indicate this and request a new and complete jaw scan. In this way, the dentist can iteratively grind the teeth or stumps until the preparations have a dimension so that the corresponding patrix cap with its minimum wall thickness fits on the tooth stump.

The exchange of information during grinding, i.e. where and how much has to be removed or ground away, can also be carried out using other preparation checking techniques. In particular, it should be mentioned that there are procedures which, in addition to the oral situation, also record the position of the contra-angle handpiece including the grinding tool and can therefore even record the change in shape of the tooth or tooth stump during the preparation of the tooth. This is displayed to the dentist on a monitor so that the preparation step can be optimized in terms of time. This means that the tooth or tooth stump only needs to be scanned again after the preparation has been completed for a final check.

Finally, either a conventional impression or an intraoral scan of the entire jaw is taken. As a result, the tooth stumps are superimposed with the corresponding patrix caps in CAD-CAM software. This results in corresponding patrix caps with a virtually prefabricated outer design (suitable for prefabricated matrices) and a corresponding inner design matched to the tooth stump (suitable for the individually ground tooth stump). These hybrid partials (prefabricated on the outside-customized on the inside) can then be produced from the green body, white body or a ready-sintered ceramic in a grinding process and then sintered if necessary. In a particularly cost-effective variant, corresponding form milling cutters are available for the milling process for the outer design of the virtual prefabricated patrices in order to optimize accuracy, surface quality and production speed. These patrices can of course also be made from a metal (titanium, zirconium, tantalum or an alloy with a main component of one of the metals, a non-precious metal alloy (e.g. chromium-cobalt-molybdenum) or a precious metal alloy. Other machining processes or additive processes, such as laser sintering or similar processes, are also suitable for production.

If a CT scan, a DVT or another 3-dimensional acquisition system (e.g. MRI) is taken prior to preparation, it is possible to record the cavities of the tooth root, i.e. the root canals, of the teeth or tooth stumps to be ground. In a particularly advantageous design, this data can be superimposed with the intraoral scan in order to avoid the need to remove so much hard tooth substance to place the selected patrix cap that the root canal is perforated. This would cause significant damage to the tooth, which would be associated with root canal treatment.

In addition, it is also possible to evaluate existing 3-dimensional data on teeth, e.g. using artificial intelligence (AI), in order to be able to make a statement about the probability of a root canal perforation using only the intraoral scan. The overall system would also be able to use artificial intelligence (AI) to anonymously document whether there has been a root canal perforation or not. This data could then be made available anonymously to all other users via the system if connected to the Internet in order to further optimize system safety.

The dental technician can thus complete the entire prosthesis on his model. The finished prosthesis can deviate from the actual patient situation in the positions of the patrices relative to the matrices in the range from, for example, 50 μm to, for example, 250 μm. The introduction of the thermo-plastic layer or the thermoplastic intermediate body provided according to one aspect of the invention is able to compensate for this. In order to be able to provide this compensation, the thermo-plastic layer should preferably have a corresponding thickness of at least 250 μm around the layer surrounding the male part. When inserting the prosthesis into the patient's mouth, the thermoplastic layer should be thermally heated above the softening point or softening interval so that it is deformable. If the prosthesis is then inserted into the patient's mouth and the patient bites down, the matrices “floating” in the softened thermoplastic layer or the softened thermoplastic intermediate body align themselves precisely with the patrices. After cooling, the thermoplastic layer is solid again and the aligned positions of the dies are retained. This process can be repeated several times as required, even after a longer wearing period.

Before the final prosthesis or prosthetics are prepared, the patrix designs to be used later are preferably determined in the CAD during the grinding of the tooth stumps in an embodiment of the process that is considered to be independently inventive. This involves selecting patrix designs already available in CAD for which corresponding matrices are available. These are aligned for an optimized insertion direction of the finished denture or prosthesis based on the data of the oral situation (residual dentition, mucosa, opposing jaw) recorded by the intraoral scanner and aligned with the ground tooth stump. This is used to plan and fabricate the cap to be produced, which forms a female part in relation to the tooth stump and a male part in relation to the denture or prosthesis, which dips into the female part of the detachable connection to the denture or prosthesis.

According to aspects of the invention, one of the three embodiments described below can be used for the dental laboratory process:
1. classic procedure with the cap already manufactured

Once the copings have been fabricated, they are temporarily attached to the tooth stumps of the physical master model (plaster model, printed acrylic model, etc.). The matrix elements are then attached to the primary crowns. The prosthesis is then planned, finished and fabricated on these. Finally, the fabricated prosthesis or prosthetics is glued or cemented to the matrix elements, preferably on the master model.
2nd procedure I with primary crown analogs integrated in the master model:

In a second variant, the corresponding prefabricated primary crowns are integrated into a printed master model. The matrix elements are then attached to the primary crowns. The prosthesis is then planned, finished and fabricated on these. Finally, the fabricated denture or prosthesis is bonded or cemented to the matrix elements, preferably on the master model.
3. procedure II with primary crown analogs integrated in the master model:

In the third variant, the primary crowns are fabricated and bonded or cemented before the master model is fabricated. After the preparation of the tooth stumps, the primary crowns are fabricated directly by the dentist (“chairside”) or in a nearby dental laboratory and bonded or cemented in the same session. An intraoral scan or classic impression can then be taken, preferably with appropriate impression caps on the primary crowns. A master model (plaster model, printed plastic model, etc.) with integrated primary crown analogs can then be fabricated. The matrix elements are then placed on the primary crowns. The prosthesis is then planned, finished and fabricated on these. Finally, the fabricated denture or prosthesis is bonded or cemented to the matrix elements, preferably on the master model

In a particularly preferred design, the thermoplastic layer or the thermoplastic intermediate body is a thermoplastic elastomer or thermoplastic silicone. The advantage is that the retention function can be better adjusted in terms of force as a snap lock. Furthermore, an elastomer layer will not fatigue so quickly and reduce the retention force. In addition, this also helps to minimize wear in the matrix-patrix system.

With regard to the shape and cross-section of the male die-matrix systems, several alternatives are conceivable in accordance with the invention. The simplest cross-sectional shape for the patrix-matrix system is considered to be round in cross-section. However, depending on the required force transmission surface, an oval design (elliptical, trioval, quadoval) may also be preferred. In the case of a round cross-section design, the space requirement increases with increasing cross-sectional diameter. The prosthesis fixed on the matrix-patrix system is also spatially limited in its ability to integrate the matrix, particularly in the buccal-palatal or buccal-lingual plane. In contrast, there is more space available in the mesio-distal extension, as the dental arch runs in this orientation. According to one aspect of the invention, oval designs can therefore be useful in order to utilize the available space particularly effectively, as these would allow a higher force transmission surface, friction surface and retention surface. The radii of the cross-sections should preferably remain as oval as possible and have neither corners, straight sections nor concavely curved areas. Furthermore, radii smaller than 1.0 mm and especially smaller than 0.5 mm are unsuitable, as higher wear is to be expected in the case of retention.

It may even be necessary to deviate from the round shape, especially when anchoring to natural teeth is planned, as it may not even be known before the actual treatment which residual teeth the different patients have. Natural teeth are usually not round in cross-section, but oval. This means that a ground tooth does not have a round cross-section and consequently a round patrix design rarely appears suitable. Rather, according to one aspect of the invention, oval/elliptical cross-sections may be advantageous and suitable in the anterior tooth region, trioval cross-sections in the canine teeth and rather quadoval cross-sections in the posterior tooth region. These can of course also have partially straight or concave areas.

According to one aspect of the invention, a geometry-related retention is provided primarily in a mesio-distal orientation so that the caps on the stumps/abutments can be designed with comparatively thin walls in the buccal-palatal or buccal-lingual plane. The space required for the retention elements in the male part preferably consists of a notched groove, which is then preferably only designed in the front and rear areas where there is sufficient space. Accordingly, the preferred shapes/designs for the retention in the matrix-patrix system are round or oval retentions, preferably circumferential, but not necessarily fully circumferential, especially in the case of non-circular cross-sections.

Preferably and according to one aspect of the invention, basic types for the patrices and patrix shapes to be considered are determined for the planning and design of the dental prosthetics according to the above explanations and made available for selection in the library.

According to an aspect of the invention which is regarded as independently inventive, when using the telescopic crowns, it is provided to heat the thermoplastic intermediate piece to a temperature above the softening temperature immediately before insertion into the patient's mouth for the purpose of adjustment and position optimization, so that it becomes correspondingly deformable. In accordance with an aspect regarded as independently inventive, the use of a heating device is provided for this purpose, which is provided in a heating area with a number of heatable contact plugs, the outer contour of which is adapted to the contour of the patrices of the telescope system. This means that such a contact plug can be inserted into the corresponding die instead of the actual male part. The respective die element can then be plugged onto one of these contact plugs for heating, and the thermoplastic intermediate body of the die element can then be heated to a temperature above its softening temperature by heating the contact plug.

The softening temperature of the thermoplastic layer or the thermoplastic intermediate body is selected above about 135° C., in particular by selecting suitable materials. This is the temperature of common and usual autoclaves with which, for example, prostheses are autoclaved in preparation for use or for disinfection. According to one aspect of the invention, the material parameters of the intermediate body are selected in such a way that thermoplastic softening does not occur at the usual temperatures during such autoclaving, so that even during such autoclaving the position of the inner cap facing the male part does not change relative to the position of the outer cap facing the denture and the position previously introduced according to the concept of the invention is also not changed during autoclaving. The lowest acceptable softening temperature for the material of the intermediate body in this sense should therefore preferably be in the range 70° C.-80° C. These are temperatures which a patient is unlikely to reach even when cleaning his denture using his own water tap, so that deposition of the inner cap facing the patrix due to deformation of the intermediate body during daily use can be ruled out.

When heating the components and immediately inserting the prosthesis for the purpose of fine alignment of the components, in particular the inner cap in relation to the outer cap, there is always a risk that the patient's mucous membrane and/or dental tissue (nerve) and/or the bone will be damaged. For this reason, the softening temperature is preferably chosen to be comparatively low. In addition, the heat capacity of the thermoplastic material forming the intermediate body should be relatively low, which is certainly the case due to the low thickness of the three-layer system.

In order to reach the softening temperature and avoid too rapid cooling before the final positioning of the inner cap facing the male part, it would be desirable to heat the entire prosthesis to the specified temperature. However, this is rather unsuitable for the intended treatment, as the prosthesis would be inserted into the patient's mouth at a temperature of at least 70° C.-80° C. in this way. Even at a temperature above 40° C.-50° C., the patient would certainly experience severe pain, and above this temperature, burns could even be expected. For this reason, according to one aspect of the invention, it is intended to heat only the intermediate body or the two-or three-layer system of the male part forming it accordingly.

In order to solve this problem, it has been found that, according to one aspect of the invention, heating should take place via the inner cap facing the male part. For this purpose, the heating device described above is particularly advantageous, which has a heating element, preferably an electric heating element, which is geometrically adapted in shape to the female part and can thus be inserted into it. Telescopic prostheses usually have two to six and preferably four abutments, each with one of the aforementioned male/female connections. The heating device is therefore preferably equipped with a plurality of, preferably six, of the aforementioned heating elements or contact plugs, so that all matrix elements of a dental prosthesis can be heated simultaneously and thus prepared for insertion.

A defined supply of thermal energy over a defined period of time enables the amount of heat required to soften the intermediate body to be introduced into it or the thermoelastic layer in order to enable fine alignment of the layer facing the patrices. These heating elements or heating patrices are preferably made of a metal, preferably with a high thermal conductivity, in particular gold, for optimized heat dissipation. They are preferably geometrically as identical as possible to the actual patrices. Small grooves in the axial direction can only be provided for easier removal, so that no negative pressure can form after heating during removal. If the thermoplastic layers are heated, removal could damage them if negative pressure is present. Furthermore, a handle is preferably formed behind the actual heating patrix so that the heating patrices can be easily inserted into the matrices and removed again.

Preferably, the heating matrices are equipped with an internal electric heater with integrated temperature control. This ensures that overheating and thus damage to the female part/patrix system or the prosthesis itself cannot occur. This control means that temperature sensors are integrated into the heating matrices. However, the heating matrices can also be equipped with a different heat source or supplied via an external heat source. It is also possible for the heating pads to merely heat the thermoelastic layer via another energy source without being heated themselves.

Preferably, the heating elements are connected to a central temperature control unit via cable connections. This preferably has four or six connections so that four or six heating elements can be controlled simultaneously. Alternatively, and depending on the intended use, such a unit could also be provided with a larger number of connections, for example 6, 8 or 10. According to one aspect of the invention, there should be at least as many as there are usually support pillars in a prosthesis. These are at least three or four, but can also be 6 to 8. Preferably, the heating cartridges are provided directly with a cable, but are connected in the direction of the control unit via a plug for removal.

In a particularly preferred design, the heating elements are equipped with rechargeable batteries, which are only located in a charging station. In this case, the temperature control unit is integrated into the heating elements.

With regard to their shape, it is advantageous for geometrically different matrix/patrix systems to also provide these different geometries for the heating matrices.

According to a further aspect of the invention, which is regarded as independently inventive, a ceramic can be provided as the material for the male part. Ceramic is particularly suitable as a male part material for aesthetic reasons alone. For example, the patient usually has a more pleasant feeling when a prosthesis is removed if the supporting abutments are tooth-colored and not metal-colored. Metals are also good heat conductors. If the thermoplastic layer or the thermoplastic intermediate body is heated to compensate for the accuracy of fit, this heat is also transferred to the patrix when it cools. In addition, with natural teeth there is a risk that the heat is transferred to teeth that are still vital, resulting in damage to the dental nerve.

Although the heat capacity of the thermoplastic layer and the patrix is preferably selected to be rather low by design, possible damage cannot be completely ruled out at present. Consequently, materials with a rather low thermal conductivity are particularly suitable as patrix materials. Metal veneers made of plastic or ceramic are therefore preferred. In particular, however, it should be mentioned that patrix caps made of solid ceramic and especially Al₂O₃ or a ZrO₂ ceramic, which are excellent heat shields, should preferably be used.

According to an aspect that is considered to be inventive in its own right, metallic particles can be embedded in the thermoplastic intermediate body. The intended heating up to the softening of the thermoplastic layer can then be effected by induction and thus possibly without contact.

According to a further aspect that is regarded as independently inventive, magnetic particles, such as iron or iron oxide, can be embedded in the thermoplastic intermediate body. The intended heating can then be carried out, in particular by generating friction, with an alternating magnetic field and thus also without contact.

According to a further aspect that is considered to be inventive in its own right, the inner cap facing the male part can be designed to “float” in the patient's mouth before insertion, i.e. instead of the thermoplastic intermediate body, a cavity can initially be provided between the inner and outer caps. The dental technician then creates an access channel, which is filled by the dentist with an adhesive or elastomer or thermoplastic material or similar during insertion.

According to the embodiments described above, the die designed according to the concept of the invention is essentially to be regarded as a three-component component (or, viewed in cross-section, as a three-layer system), which comprises the components inner cap, outer cap and intermediate body arranged between these. According to a further aspect of the invention, a further, i.e. fourth, component or layer may additionally be provided. The fourth layer according to this aspect of the invention may be an outer fourth layer or component, which in turn surrounds the outer cap on the outside and enables a detachable connection of the matrix element to the prosthesis or dental prosthesis. This allows the matrix element to be mechanically mounted on the prosthesis or dental prosthesis and easily removed again without destruction. This makes it possible to initially anchor the matrix or cap firmly in the prosthesis and, if necessary, e.g. after several years of wear, to remove it relatively easily and replace it with a new multi-part matrix system. This makes it particularly easy to replace an inserted female part, for example when the female part starts to wear. It is particularly important that the adhesive force in this system is significantly higher than that of the matrix-patrix system. It should be a type of ratchet that damages or destroys the die to be replaced during replacement. A special tool in the form of pliers or similar would be useful.

In a further embodiment, which is regarded as independently inventive, the multi-part die element can have an intermediate body made of light-curing plastic instead of or in addition to the thermo-plastic intermediate body. This could, for example, be incorporated in an uncured, i.e. still deformable, state in accordance with the procedure described above, so that the inner and outer caps of the die can be suitably aligned with one another. Once this has been done, the intermediate body could be cured using UV light, for example, so that the position recorded is fixed.

The advantages achieved with the invention consist in particular in the fact that completely pre-fabricated matrix-patrix systems can be produced on natural teeth in the manner according to the invention, in particular with a matrix that can be produced in a CAD-Cam process from metal and preferably from a ceramic. Furthermore, a system with reproducible adhesive force that is hardly dependent on the chewing force can be provided in this way. The system exhibits very little wear, particularly due to the extremely high accuracy of fit that can be achieved. High-precision fits for high wearing and chewing comfort can be achieved, and handling is simple and uncomplicated, i.e. the dental technician can fix the female part in the denture with minimized accuracy of fit in the patient's mouth. Furthermore, the system has a particularly small footprint.

An embodiment of the invention is explained in more detail with reference to a drawing. It shows:

FIG. 1 schematic of a telescopic system for fixing a dental prosthesis in the oral cavity of a patient,

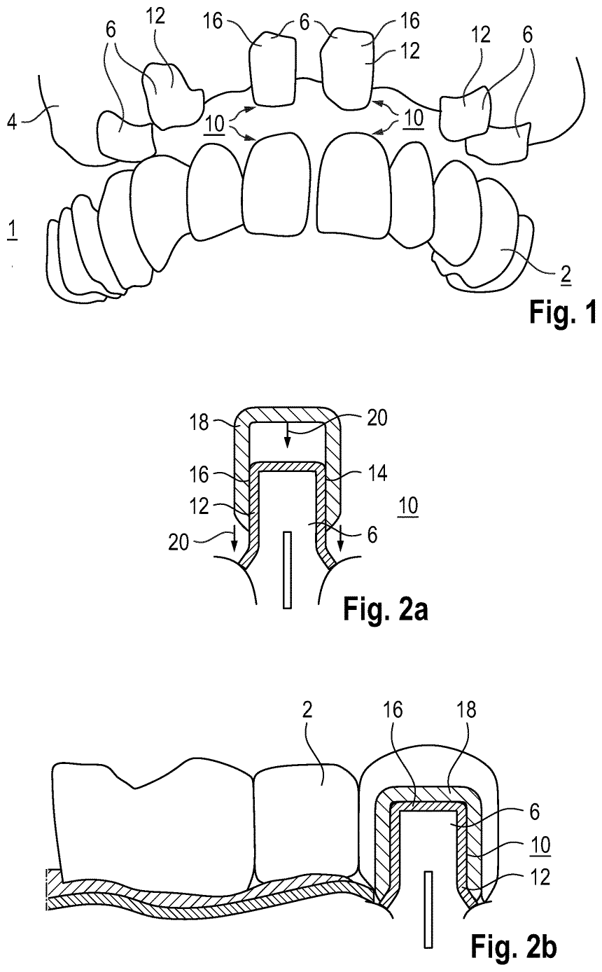

FIG. 2 longitudinal section of a telescopic crown of the system according to FIG. 1,

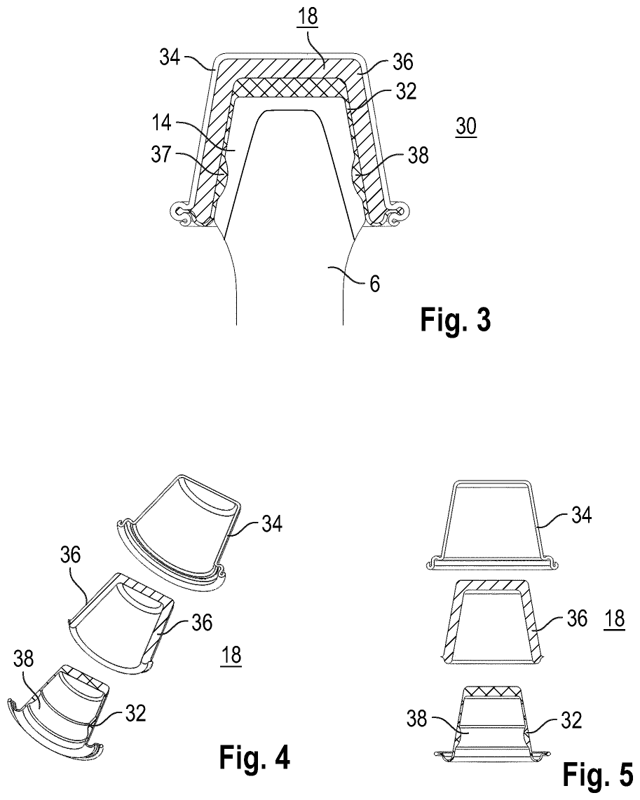

FIG. 3 a telescopic crown according to one aspect of the invention in longitudinal section,

FIG. 4 an exploded view of a matrix element of the telescopic crown according to FIG. 3 in partial perspective section,

FIG. 5 the matrix element of the telescopic crown according to FIG. 3 in exploded view in longitudinal section,

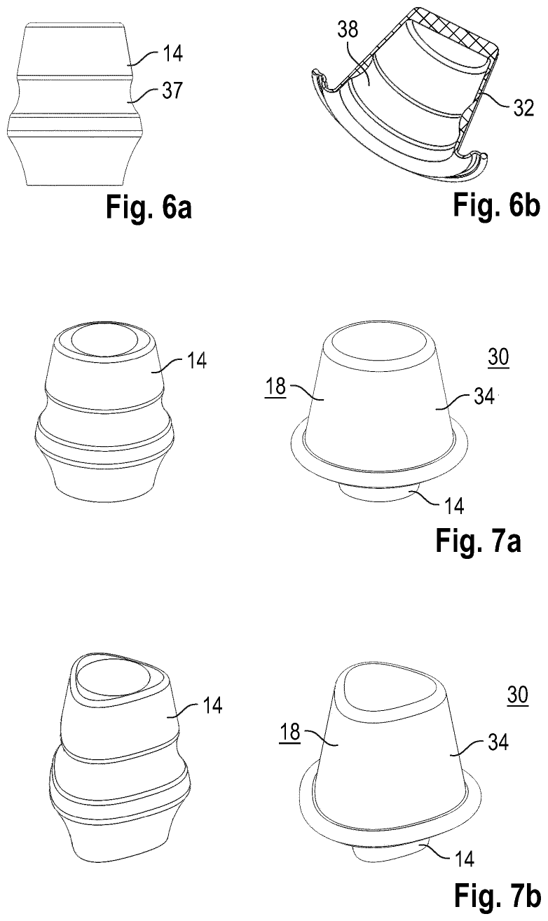

FIG. 6 a lateral view of a male element of the telescopic crown according to FIG. 3 and an inner cap of the female element according to FIGS. 4, 5, the inner contour of which is adapted to the outer contour of the male element,

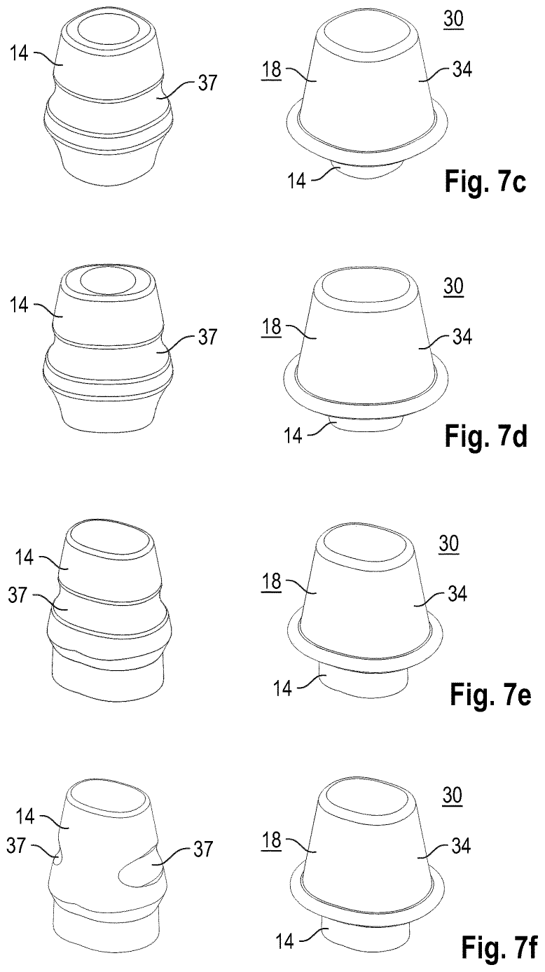

FIG. 7 in each case in pairs a male element and the associated attached female element in different cross-sectional geometries,

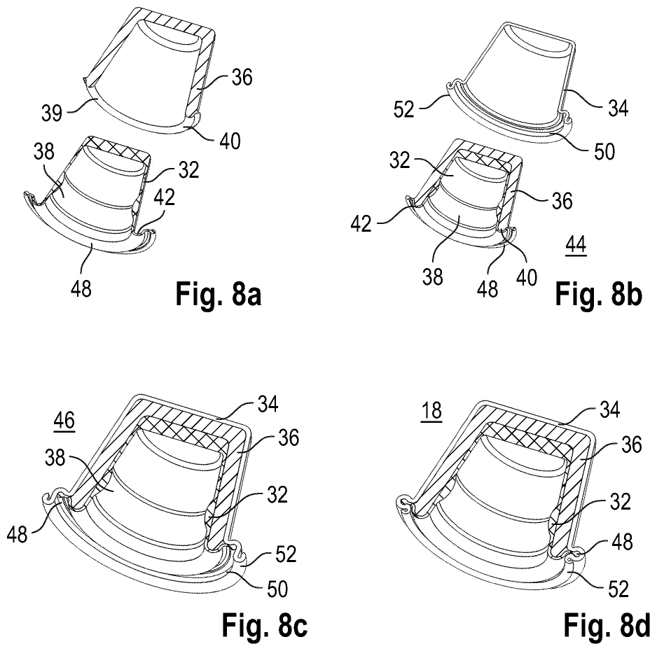

FIG. 8 a sequence of assembly steps of the die element according to FIGS. 4, 5 in partial section in perspective view,

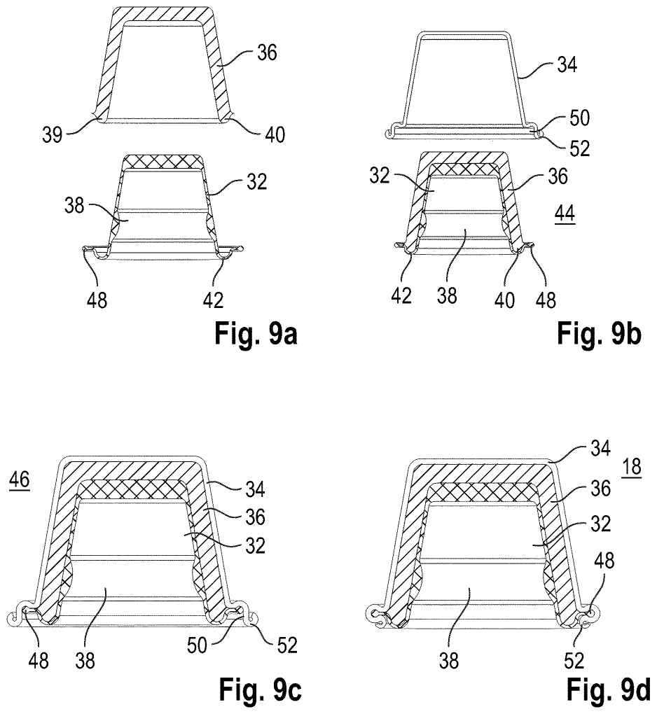

FIG. 9 a sequence of assembly steps of the die element according to FIGS. 4, 5 in longitudinal section,

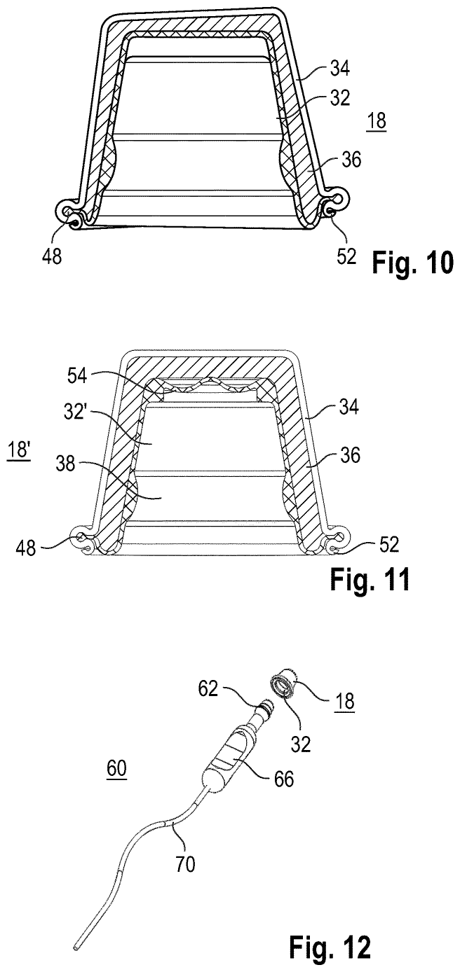

FIG. 10 the die element according to FIG. 9d with “tilted” outer cap,

FIG. 11 an alternative embodiment of a die element in longitudinal section,

FIG. 12 section of a heater, and

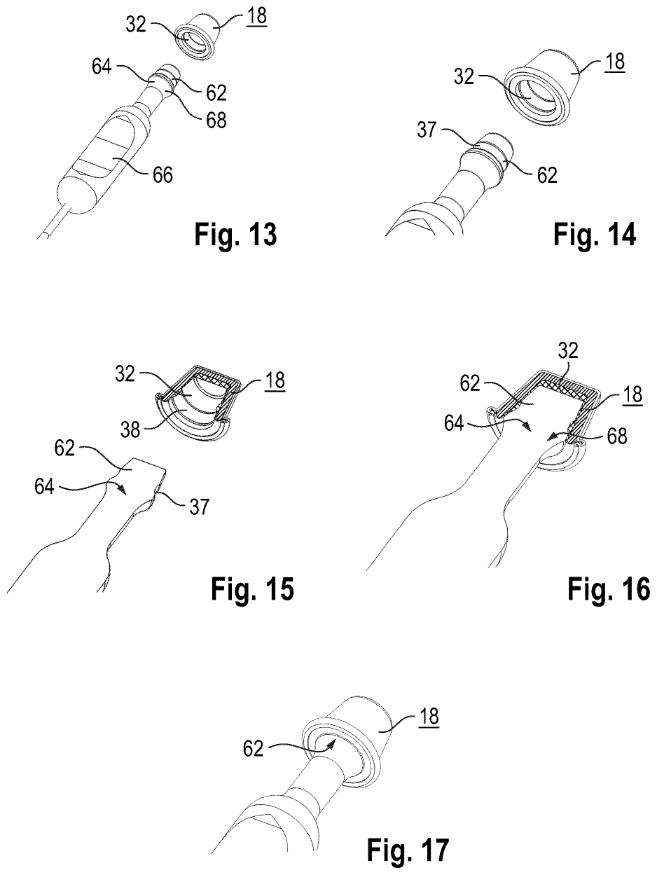

FIG. 13-17 enlarged representation of a contact plug of the heater according to FIG. 12.

Identical parts are marked with the same reference signs in all figures.

The telescopic system 1 according to FIG. 1 is used for the removable attachment of a dental prosthesis 2 in the oral cavity of a patient. In the embodiment example, the dental prosthesis 2 shown is a complete upper jaw prosthesis; alternatively, however, other prostheses such as a bridge, for example, which closes a gap between several teeth of a residual dentition, or individual prostheses for replacing a single tooth could of course also be provided. The dental prosthesis 2 is designed to be detachably connected to a number of supporting pillars that are fixed to the upper jaw 4 and thus arranged in the patient's oral cavity. In the embodiment example shown, the supporting abutments are teeth 6 of a residual dentition that remain in the patient's oral cavity and are suitably ground on their surface.

The telescopic system 1 comprises a number of so-called telescopic crowns 10—in the embodiment example corresponding to the number of ground teeth 6 of the residual dentition—with which the dental prosthesis 2 is removably fixed to the upper jaw 4 and thus in the oral cavity of the patient. Such a telescopic crown 10, as shown in conventional design in longitudinal section in FIG. 2 a in single design and in FIG. 2 b in longitudinal section as fastening means for the dental prosthesis 2, essentially represents a double-crown system. On the one hand, this comprises a “lower” or base crown 12, also referred to as a “primary crown”, which is firmly attached to a suitably prepared, for example ground, tooth 6 in the manner of a conventional crowning. The primary crown 12, which is also visible in the representation according to FIG. 1 for the teeth 6 shown there, is shown in the representations according to FIG. 2 in the state attached to the respective tooth 6.

The primary crown 12 is designed in the usual manner as a male element 14, which forms a contact pin 16 on its surface. An “upper” crown segment or matrix element 18, also referred to as a “secondary crown”, can be detachably or removably attached to the male element 14 as a second essential component of the telescopic crown, for example by clicking it on or attaching it. In FIG. 2 a, the process of pushing on is indicated by the arrows 20, whereas in FIG. 2 b, the telescopic crown 10 is shown in the state with the matrix element 18 completely pushed onto the male element 14. In the type of matrix-patrix system of the telescopic crown 10 formed by the crown segments 14, 18, the “upper” crown segment 18 forming the matrix serves as a support for the dental prosthesis 2, which is suitably firmly connected to the matrix elements 18.

In telescopic systems 1 of the type described, the accuracy of fit of the dental prosthesis 2 in the patient's mouth is a significant factor, as a high degree of accuracy of fit is a prerequisite for a tight fit of the prosthesis 2 in the patient's mouth, for example. Furthermore, a high accuracy of fit naturally also requires a high level of wearing comfort for the patient so that they can feel as comfortable as possible with the prosthesis 2. The more accurately the prosthesis is manufactured, the more securely it can fit in the patient's mouth, which in turn leads to a very high level of wearing and chewing comfort. However, with regard to conventional manufacturing methods, in which the tooth situation in the patient's mouth is usually first determined and then transferred to a physical or virtual model, which is then used to plan and manufacture the restoration, inaccuracies in the fit are to be expected in an undesirable manner.

In order to take this into account, according to one aspect of the present invention, a construction method for a telescopic crown 30 is provided, as shown in the longitudinal section in FIG. 3, and which permits a particularly accurate production of the double crown system. The design of the telescopic crown 30 according to the invention is based on the concept of manufacturing the essential components of the double crown system with an accuracy that is considered acceptable and then, after pre-assembly of the components, inserting them into the patient's mouth with a certain degree of flexibility and malleability and fitting them there, in response to the actual oral situation and/or taking into account current data characteristic of the intraoral oral situation and the restoring and shearing forces occurring during insertion, to allow the final positioning of the components adapted to the actual oral situation and then to fix them in place.

For this purpose, the telescopic crown 30 according to the invention, shown in FIG. 3, comprises as essential components, comparable to the conventional design of the telescopic crown 10, on the one hand a male element 14 provided for mounting or crowning on a residual tooth 6 and on the other hand a corresponding female element 18 which can be plugged onto the male element 14 and can be firmly connected to the dental prosthesis 2. In contrast to the conventional embodiment in the telescopic crown 10, however, according to one aspect of the present invention, the matrix element 18 in the telescopic crown 30 is made in several parts and comprises an inner cap 32 which can be slipped onto the male element 14 and an outer cap 34 which can be attached to the dental prosthesis 2, an intermediate body 36 made of thermoplastic material connecting them being arranged in the space between the inner and outer caps 32, 34. This multi-part design of the matrix element 18 is particularly clearly recognizable from the exploded view in the perspective partial section in FIG. 4 and in the longitudinal section in FIG. 5.

This multi-part design using a temporarily deformable intermediate body 36 or a temporarily deformable intermediate layer makes it possible for final positioning and alignment of the components in relation to each other, i.e. in particular of the inner cap 32 and outer cap 34, to take place only during insertion of the telescopic crown 30 into the oral cavity. The outer cap 34 can therefore already be firmly mounted on the dental prosthesis 2 during the preparation for insertion. For the insertion, it is then intended to heat the thermoplastic intermediate body 36 beforehand to above its softening temperature so that it is malleable. In this state, the incorporation can then be carried out so that the inner and outer caps 32, 34 are suitably aligned with one another in accordance with the fine details of the oral situation and in response to the resulting pressure and positioning forces, with deformation of the intermediate body 36. The alignment of the inner cap 32 relative to the outer cap 34 and thus the final positioning is thus adapted to the actual conditions in the oral cavity. Subsequently, the intermediate body 36 can cool down while retaining the assumed shape and thus maintaining the underlying position, and consequently solidify again. After solidification, the optimized alignment of the components recorded in this way is thus fixed.

The dental prosthesis 2 can thus be manufactured by heating the intermediate body 36 to a temperature above its softening temperature and thus making it deformable in order to align the respective inner cap 32 precisely relative to the outer cap 34 of the respective matrix element 18, and then, using the deformability of the intermediate body 36, the inner cap 32 is aligned relative to the outer cap 34 with regard to an optimized accuracy of fit, taking into account intraoral data reflecting the actual dentition situation in the patient's oral cavity, before the relative position of the inner cap 32 relative to the outer cap 34 set in this way is preserved as a result of the solidification of the intermediate body 36 occurring during the subsequent cooling of the intermediate body 36

According to one aspect of the invention, the intermediate body 36 is preferably specifically adapted to the usual handling processes for use in dental treatments with regard to its choice of material and its material parameters. In particular, it is advantageously taken into account that autoclaving is common and widely used in such processes. According to one aspect of the invention, the material parameters of the intermediate body 36 are selected such that thermoplastic softening does not occur at the usual temperatures during such autoclaving, so that even during such autoclaving the position of the inner cap 32 facing the male part 14 does not change relative to the position of the outer cap 34 facing the dental prosthesis 2 and the position previously introduced according to the concept of the invention is not changed during autoclaving either. The softening temperature of the thermoplastic intermediate body 36 is selected above about 135° C., in particular by suitable material selection. This is the temperature of common and usual autoclaves with which, for example, prostheses are autoclaved in preparation for use or for disinfection.

The patrix element 14 of the telescopic crown 30 is shown in FIG. 6 a in lateral view. In the embodiment example, the patrix element 14 has a substantially round cross-section, although it can alternatively also be designed with non-circular cross-sections, for example oval, elliptical, trioval or the like, preferably adapted to the geometric conditions at the insertion site in the oral cavity. As can be clearly seen from the illustration in FIG. 6, the patrix element 14 has a circumferential groove 37 which, in conjunction with an associated inner bead 38 on the inside of the inner cap 32, ensures retention when the inner cap 32 is applied and thus provides additional fixation. The connection system can thus be designed in the manner of a snap-on or latching connection, in which the die element 18 can be clicked or snapped onto the male element 14 via its inner cap 32. As can be seen from the illustration in FIG. 6 b, the inner contour of the inner cap 32 is adapted to this.

According to one aspect of the invention, a method is provided for manufacturing the dental prosthesis 2 in which, on the basis of intraoral data reflecting the actual dentition situation in the patient's oral cavity, those male element basic types 14 are selected from a number of male element basic types stored in a component library which permit reliable fixation of the prosthesis 2 with the least possible removal of tooth structure, and the required grinding of the teeth 6 is determined on the basis of this selection and made available to the practitioner as instructions for action.

Depending on the details of the oral situation, different basic types can be considered for the patrix elements 14, which are provided for the practitioner to choose from. FIG. 7 shows an example of a number of geometric variants of the patrix element 14 in combination with the respective associated matrix element 18 with differently designed cross-sectional contours, as they can be selected as required depending on the insertion site and the patient's oral situation, in perspective view. The male element 14 on the one hand and the complete telescopic crown 30, i.e. the female element 18 placed on the male element 14, are each shown together in pairs. In particular:
FIG. 7 a a male and female element 14, 18 with a non-round, oval or elliptical cross-section,
FIG. 7 b a male and female element 14, 18 with a trioval cross-section,
FIG. 7 c a male and female element 14, 18 with a square oval cross-section,
FIG. 7 d a male and female element 14, 18 with an elongated rectangular cross-section with rounded corners to fulfill the ovality criteria,
FIG. 7 e a male and female element 14, 18 with a comparatively “flat”, elongated cross-section,
FIG. 7 f the male and female element 14, 18 according to FIG. 7 e with additional, partially circumferential undercut or groove 37 to provide additional retention.

The assembly of the die element 18 from the prefabricated components is shown in FIG. 8 by means of a sequence of assembly steps in partial section in perspective view and in FIG. 9 by means of a sequence of assembly steps in longitudinal section. First, as shown in FIGS. 8a, 9a, the inner cap 32 is inserted into the intermediate body 36. This has a circumferential end bead 40 at its free end 39, which is inserted into a receiving groove 42 formed on the end of the inner cap 32. The resulting partially assembled ensemble 44 is inserted into the outer cap 34, as shown in FIGS. 8 b, 9 b, so that the ensemble 46 shown in FIGS. 8 c, 9 c is formed. A circumferential fixing rim 48 formed in the end region of the inner cap 32 is inserted into a receiving ring 50 of the outer cap 34. Subsequently, a flanged edge 52 surrounding this is folded over to form a crimped or flanged connection, so that it surrounds the fixing edge 48 as shown in FIGS. 8 d, 9 d and thus adequately fixes the inner cap 32 to the outer cap 34 in the sense of a pre-assembly.

As mentioned above, the matrix element 18 constructed and pre-assembled in this way is heated during incorporation, so that the thermoplastic intermediate body 36 is heated to above its softening temperature and thus becomes deformable. The incorporation can then be carried out in this state, so that the inner and outer cap 32, 34 are suitably aligned with one another in accordance with the fine details of the oral situation and in response to the pressure and positioning forces caused thereby, with deformation of the intermediate body 36. As a result of this alignment, the inner cap 32, starting from the initially approximately parallel alignment as shown in FIG. 9 d, is tilted or otherwise repositioned relative to the outer cap 34, whereby the intermediate body 36 is deformed accordingly. The result of such a deformation, which is accompanied by the final alignment of the components, is shown by way of example in FIG. 10 by means of a longitudinal section of the die element 18 in the “tilted” state.

Compared to the initial position shown in FIG. 9 d, the deformation of the intermediate element 36 that has occurred is clearly recognizable. However, it can also be seen that the inner cap 32 is also clearly deformed in the region of the fixing edge 48 forming its base. In accordance with an aspect which is regarded as independently inventive, this intended deformation of the inner cap 32 is taken into account by a suitable choice of material. The base region or fixing edge 48 of the inner cap 32, which forms a type of membrane, should in fact offer as little or no restoring force as possible against this deformation. According to one aspect of the invention, this is achieved by a suitable choice of material for the inner cap 32, at least in the region of the fixing edge 48. Preferably, the inner cap 32 is therefore made of a high-performance plastic, preferably PEEK.

In FIG. 11, an alternative embodiment of a die element 18′ is shown in longitudinal section. In this variant, the cover area 54 of the inner cap 32′ is corrugated, so that a certain deformability is provided, particularly in the longitudinal direction. Such an embodiment thus enables, to a certain extent, compensation or equalization of the positions of the components relative to one another in the longitudinal direction.

Taking these options into account, the dentist can make a preselection of the basic types provided in the library and then determine which combination of patrix elements can be used to minimize the overall grinding requirement, i.e. the loss of tooth structure. The prosthesis can then be planned as a whole on this basis, and the dentist can be provided with the information required for the grinding, which he can use to prepare the remaining dentition for crowning with the patrix elements 14.

In order to enable the conceptually intended heating of the thermoplastic intermediate piece 36 of the telescopic crowns 30 to a temperature above the softening temperature immediately before incorporation, a heating device 60 is provided in accordance with one aspect of the invention, which is shown in section in FIG. 12. The heating device 60 comprises a number of heatable contact plugs 62, which correspond in their outer contour to the contour of the male elements 14 of the telescopic crowns 30 and can thus be inserted into the female elements 18 or their inner cap 32 instead of the male elements 14, and of which only one is shown in FIG. 12. The contact plugs 62 are thus preferably geometrically as identical as possible to the actual male elements 14. According to one aspect of the invention, small grooves can be provided in the outer skin of the contact plugs 62 in the axial direction only for easier removal of the heated matrix elements 18, so that no negative pressure can form after heating during removal. If the thermoplastic intermediate bodies 36 are heated, removal could lead to damage to them if negative pressure is present.

The contact plugs 62 can therefore also be referred to as “heating cartridges”. For optimized heat dissipation, they are preferably made of a metal, preferably with a high thermal conductivity, in particular gold. For heating, the respective die element 18 can then be plugged onto one of these contact plugs 62, and then the thermoplastic intermediate body 36 of the die element 18 can be heated to a temperature above its softening temperature by heating the contact plug 62. The heating device 60 is equipped with a number, preferably six, of said heating elements or contact plugs 62 corresponding to the number of matrix elements 18 provided in the respective dental prosthesis 2 or the number of matrix elements 18 usually used in such dental prostheses 2, so that all matrix elements 18 of a dental prosthesis 2 can be heated simultaneously and thus prepared for insertion.

The possibility of inserting each of the contact plugs 62 into the inner cap 32 of a die element 18 becomes particularly clear from the various enlarged representations in FIGS. 13 to 17.

In the embodiment example, the heatable contact plugs 62 are provided with an integrated heating element 64 with integrated temperature control. Using a defined supply of thermal energy over a defined period of time, it is thus possible to introduce the amount of heat required for softening the intermediate body 36 into this or the thermoelastic layer in order to enable the fine alignment of the inner cap 32 facing the male part elements 14 relative to the outer cap 34 connected to the prosthesis 2. Furthermore, a handle 66 is preferably formed behind the actual heating patrix 62 in order to be able to easily insert the heating patrices 62 into the matrix elements 18 and remove them again.

The heating elements 64, designed as internal electrical heating, ensure that overheating and thus damage to the matrix-patrix system or the prosthesis 2 itself cannot occur. This control requires that temperature sensors 68 are integrated in the heating matrices 62. However, the heating patrices 62 could alternatively also be equipped with a different heat source or be supplied via an external heat source. In addition, it is also possible that the heating elements 62 merely heat the thermoelastic intermediate body 36 via another energy source without being heated themselves.

According to one aspect of the invention, a plurality of the heating patrices 62 are each connected via a cable connection 70 to a common, central temperature control unit not shown in detail in FIG. 12. This preferably has four or six connections in order to be able to control four or six heating cartridges 62 simultaneously. Alternatively, and depending on the intended use, such a unit could also be provided with a larger number of connections, for example 6, 8 or 10. According to one aspect of the invention, there should be at least as many as there are usually support pillars provided in a prosthesis 2. Preferably, the heating patrices 62 are provided directly with a cable connection 70, but are detachably connected to the central control unit in the direction of the latter via a plug.

In a particularly preferred embodiment, the heating elements 62 are equipped with rechargeable batteries, which are only located in a charging station. In this case, the temperature control unit is integrated into the heating elements 62.

### LIST OF REFERENCE SYMBOLS

1 Telescope system
2 Denture
4 Upper jaw
6 Tooth
10 Telescopic crown
12 Primary crown
14 Patrix element
16 Contact pin
18 Matrix element
20 Arrow
30 Telescopic crown
32 Inner cap
34 Outer cap
36 Intermediate body
37 Groove
38 Inner bead
39 End
40 Bead
42 Recording groove
44, 46 Ensemble
48 Fixing edge
50 Recording ring
52 Flared edge
54 Lid area
60 Heater
62 Contact plug
64 Heating element
66 Handle element
68 Temperature sensors
70 Cable connection

1. A method for producing a dental prosthesis which, for removable fixation on a number of teeth in the mouth of a patient, each crowned with a respective male element is provided with a number of matrix elements each corresponding to one of the male elements wherein the or each matrix element is made in several parts and comprises an inner cap which can be fitted onto the associated male element and an outer cap attached to the dental prosthesis, and wherein an intermediate body made of thermoplastic material is arranged in the space between the inner and outer caps, and connects them to one another, in which the intermediate body is heated to a temperature above its softening temperature and thus made deformable in order to align the inner cap precisely relative to the outer cap of the respective matrix element, and then, using the deformability of the intermediate body, the inner cap is aligned relative to the outer cap with regard to an optimized accuracy of fit, taking into account intraoral data reflecting the actual dentition situation in the oral cavity of the patient, before the relative position of the inner cap set in this way relative to the outer cap is preserved as a result of the solidification of the intermediate body occurring during the subsequent cooling of the intermediate body.

2. A method for producing a dental prosthesis which, for removable fixation on a number of teeth in the mouth of a patient, each crowned with a respective male element is provided with a number of matrix elements each corresponding to one of the male elements, in particular using multi-part matrix elements which each comprise an inner cap which can be fitted onto the associated male element and an outer cap which is attached to the dental prosthesis, wherein in the space between the inner and outer caps, an intermediate body made of thermoplastic material connecting them to one another being arranged in the space between the inner and outer caps, in particular according to claim 1, wherein from a number of basic male element types stored in a component library, on the basis of intraoral data reflecting the actual dentition situation in the patient's oral cavity, those male elements are selected which permit reliable fixation of the prosthesis with the least possible removal of tooth structure, and the required grinding of the teeth is determined on the basis of this selection and made available to the practitioner as instructions for action.

3. A method for producing a dental prosthesis which, for fixation on a number of teeth in the mouth of a patient, each crowned with a respective male element, is provided with a number of matrix elements each corresponding to one of the male elements, wherein the or each matrix element is made in several parts and comprises an inner cap which can be fitted onto the associated male element and an outer cap attached to the dental prosthesis, and wherein an intermediate body made of thermoplastic material is arranged in the space between the inner and outer caps and connects them to one another, in which the intermediate body is heated to a temperature above its softening temperature and thus made deformable in order to align the inner cap precisely relative to the outer cap of the respective matrix element, and then, using the deformability of the intermediate body, the inner cap is aligned relative to the outer cap with regard to an optimized accuracy of fit, taking into account intraoral data reflecting the actual dentition situation in the oral cavity of the patient, before the relative position of the inner cap set in this way relative to the outer cap is preserved as a result of the solidification of the intermediate body occurring during the subsequent cooling of the intermediate body.

4. A dental prosthesis which, for removable fixation on a number of teeth in the mouth of a patient, each crowned with a respective male element, is provided with a number of matrix elements each corresponding to one of the male elements, wherein the or each matrix element is each made in several parts and comprises an inner cap which can be fitted onto the associated male element and an outer cap attached to the dental prosthesis, and wherein an intermediate body of thermoplastic material connecting the inner and outer caps is arranged in the space between them, obtained by a process in which the intermediate body is heated to a temperature above its softening temperature and thus made deformable in order to align the inner cap precisely relative to the outer cap of the respective matrix element, and then, using the deformability of the intermediate body, the inner cap is aligned relative to the outer cap with regard to an optimized accuracy of fit, taking into account intraoral data reflecting the actual dentition situation in the oral cavity of the patient, before the relative position of the inner cap set in this way relative to the outer cap is preserved as a result of the solidification of the intermediate body occurring during the subsequent cooling of the intermediate body.
